(19) United States
(12) Patent Application Publication
(10) Pub. No.: US 2011/0218608 A1
(43) Pub. Date: Sep. 8, 2011

(54) Vascular Prosthesis Delivery System and Method

(75) Inventors: Christopher P. Cheng, Palo Alto, CA (US); Eric Hsiang Yu, Moraga, CA (US); Eric W. Leopold, Redwood City, CA (US); Ethan A. Gilbert, San Jose, CA (US)

(73) Assignee: NovoStent Corporation, Mountain View, CA (US)

(21) Appl. No.: 12/879,436

(22) Filed: Sep. 10, 2010

Related U.S. Application Data
(60) Provisional application No. 61/241,345, filed on Sep. 10, 2009.

(51) Int. Cl. A61F 2/84

(52) U.S. Cl. 623/1.11

(57) ABSTRACT

A vascular prosthesis delivery system comprises a radially self expandable vascular prosthesis and a delivery sheath with a lumen with a smaller diameter storage region and a larger diameter delivery region. A prosthesis is housed within the storage region and is movable into the delivery region for delivery at a target site within a patient. The delivery force required to move the prosthesis from the delivery region into the patient can be less than the force required to move the prosthesis from the storage region into the delivery region. In some examples, the storage region defines a tapered lumen expanding in diameter in a distal direction. In some examples, the storage and delivery regions are generally coextensive and define a tapered lumen expanding in diameter in a distal direction. A method stores a vascular prosthesis in the storage region and delivers it to a target site from the delivery region.

### CROSS-REFERENCE TO OTHER APPLICATIONS

This application claims the benefit of U.S. provisional patent application No. 61/241,345 filed 10 Sep. 2009, the disclosure of which is incorporated by reference.

### BACKGROUND OF THE INVENTION

Today, there are a wide range of intravascular prostheses on the market for use in the treatment of aneurysms, stenosis, and other vascular disorders. Stents, stent grafts, and other vascular prostheses are well known for treating a myriad of diseases and illnesses in vasculature. For percutaneous interventions, many vascular prostheses are inserted into the body within a catheter and accurately and safely deployed at the desired treatment site.

Previously known self-expanding vascular prostheses can be retained in a catheter delivery configuration using an outer sheath; the prosthesis then self-expands when the outer sheath is retracted. See, for example, US patent application publication number US 2008/0021657 A1, assigned to the assignee of this application. Due to this configuration, several potentially undesirable effects are present during deployment of the prosthesis. Because the outer sheath is restraining the prosthesis, the frictional force between the prosthesis and outer sheath must be overcome to deploy the stent. The frictional force may be prohibitive to sheath withdrawal, and may shift the position of the prosthesis. Alternatively, self-expanding vascular prostheses can be secured to the outer surface of a delivery catheter; the prosthesis is then released from the delivery catheter at the target site within the patient. See, for example, U.S. Pat. Nos. 5,772,668 and 6,514,285.

This application is directed to systems in which self expanding vascular prosthesis are retained in their radially contracted states through the use of an outer delivery sheath. Portions of the vascular prosthesis may be secured to an inner delivery catheter, as in US 2008/0021657 A1, or an inner delivery catheter may not be used.

It is typically desirable that the vascular prosthesis have a high outward acting force to improve in vivo performance. However, this high outward acting force can result in a high frictional force during deployment, and requires the outer sheath, sometimes called the outer delivery sheath, to be strong both radially and longitudinally. A high deployment force is undesirable from safety, ergonomic, and control perspectives, e.g. placement accuracy. A high deployment force requires the use of stronger materials and/or a thicker outer sheath. These material and dimensional constraints are undesirable; the stronger materials are often more expensive and less flexible than traditional materials, and a thicker outer sheath moreover results in a larger device profile. Additionally, with a high deployment force, the outer sheath is more likely to stretch and neck down, resulting in additional deployment difficulties.

The vascular prosthesis is generally restrained in the outer sheath from the time the vascular prosthesis is loaded, packaged, sterilized, transported, and then deployed by the end-user. The device must remain operational following exposure to all of these environments, which can vary dramatically in temperature, humidity, and mechanical impact. Throughout these different environments, the self-expanding vascular prosthesis maintains a residual outward acting force. The changes in humidity and temperature can cause changes in the dimensions and physical properties of the device, resulting in undesirable deployment characteristics of the device. For example, sterilization through the use of ethylene oxide gas is a common sterilization procedure that requires elevated temperatures and high humidity to adequately sterilize the device. These conditions may cause the materials used in the device to expand and weaken, allowing the vascular prosthesis to expand radially and embed into the outer sheath, resulting in higher deployment forces and potential increases in profile. Additionally, the prosthesis material may have material properties such that elevated temperature results in the vascular prosthesis exerting a higher outward force against the outer sheath causing a further likelihood of higher deployment forces.

### BRIEF SUMMARY OF THE INVENTION

One example of a vascular prosthesis delivery system comprises a radially self expandable vascular prosthesis and a delivery sheath. The delivery sheath has a lumen. The lumen has a smaller diameter storage region and a larger diameter delivery region. A vascular prosthesis is housed within the storage region and is movable into the delivery region for delivery of the vascular prosthesis at a target site within a patient. The delivery force required to move the vascular prosthesis from the delivery region into the patient can be less than the force required to move the vascular prosthesis from the storage region into the delivery region. In some examples, the delivery sheath comprises a main delivery sheath and an extension cartridge mountable to the main delivery sheath, the extension cartridge comprising the smaller diameter storage region. In some examples, the smaller diameter storage region and the larger diameter delivery region are each generally constant diameter regions. In some examples, the storage region defines a tapered lumen, the tapered lumen expanding in diameter in a distal direction. In some examples, the storage and delivery regions are generally coextensive and define a tapered lumen expanding in diameter in a distal direction.

One example of a method for storing a vascular prosthesis and delivering the vascular prosthesis to a target site within a patient comprises the following. A vascular prosthesis delivery system is obtained, the vascular prosthesis delivery system comprising radially self expandable vascular prosthesis stored within a smaller diameter storage region of a lumen of a delivery sheath. The vascular prosthesis is moved from the smaller diameter storage region into a larger diameter delivery region of the lumen of the delivery sheath. The vascular prosthesis is moved from the larger diameter delivery region to a target site within a patient. Whereby the force required to move the vascular prosthesis from the delivery region into the patient can be less than the force required to move the vascular prosthesis from the storage region into the delivery region. In some examples, the obtaining step is carried out with the delivery sheath comprising a main delivery sheath and an extension cartridge mountable to the main delivery sheath, the extension cartridge comprising the smaller diameter storage region; this example further comprises removing the extension cartridge from the main delivery sheath after the vascular prosthesis is moved from the storage region to the delivery region. In some examples, the obtaining step is carried out with the smaller diameter storage region defining a tapered smaller diameter storage region, the tapered smaller diameter storage region expanding in diameter in a distal direction, and wherein the first moving step is carried out with the vascular prosthesis being moved from the tapered smaller diameter storage region into the larger diameter storage region. In some examples, the obtaining step is carried out so that the storage and delivery regions comprise a generally coextensive storage/delivery region, the storage/delivery region being a tapered storage/delivery region expanding in diameter in a distal direction.

Other features, aspects and advantages of the present invention can be seen on review of the drawings, the detailed description, and the claims which follow.

### BRIEF DESCRIPTION OF THE DRAWINGS

FIGS. 1 and 2 are side views of a vascular prosthesis having alternating helical portions shown in radially expanded and radially contracted states;

FIG. 3 illustrates a vascular prosthesis delivery system suitable for use with the vascular prosthesis of FIGS. 1 and 2;

FIG. 4 shows an atraumatic tip at the distal end of the delivery catheter of FIG. 3;

FIGS. 5 and 5A show an alternative embodiment of the vascular prosthesis of FIGS. 1 and 2 shown with the body in a flattened state and a radially contracted state, respectively;

FIG. 6 shows a vascular prosthesis delivery system of the type including a cartridge which houses the vascular prosthesis, the cartridge being mountable to an end of the outer delivery sheath;

FIG. 7 shows the outer delivery sheath of FIG. 6 after the vascular prosthesis has been placed into the interior of the outer delivery sheath and the cartridge has been removed;

FIG. 8 shows another example of the invention in which the vascular prosthesis has been placed within a smaller diameter storage region of an outer sheath for storage and sterilization;

FIG. 9 shows the outer delivery sheath of FIG. 8 with the vascular prosthesis moved from the storage region to the adjacent distal larger diameter region prior to delivery of the vascular prosthesis to the target site within the patient;

FIG. 10 shows a further example of the invention in which the outer delivery sheath has a tapering lumen and the prosthesis is within the smaller diameter proximal region for storage and sterilization;

FIG. 11 shows the outer delivery sheath from FIG. 10 with the vascular prosthesis moved to the distal larger diameter region prior to delivery of the vascular prosthesis to the target site within the patient; and

FIG. 12 shows a further example of the invention in which the outer delivery sheath has a tapering lumen in which the vascular prosthesis resides until delivery of the vascular prosthesis to the target site within the patient.

### DETAILED DESCRIPTION OF THE INVENTION

The following description will typically be with reference to specific structural embodiments and methods. It is to be understood that there is no intention to limit the invention to the specifically disclosed embodiments and methods but that the invention may be practiced using other features, elements, methods and embodiments. Preferred embodiments are described to illustrate the present invention, not to limit its scope, which is defined by the claims. Those of ordinary skill in the art will recognize a variety of equivalent variations on the description that follows. Like elements in various embodiments are commonly referred to with like reference numerals.

One aspect of the present invention is the recognition of the drawbacks of previously known devices created by the vascular prosthesis exerting an outward radial force on the outer delivery sheath, discussed above, which causes embedding of the vascular prosthesis into the outer delivery sheath with the resultant increased and unpredictable delivery force. It would be desirable to provide an implantable vascular prosthesis delivery system with optimal delivery flexibility and profile, a low, predictable deployment force, and accurate vascular prosthesis placement.

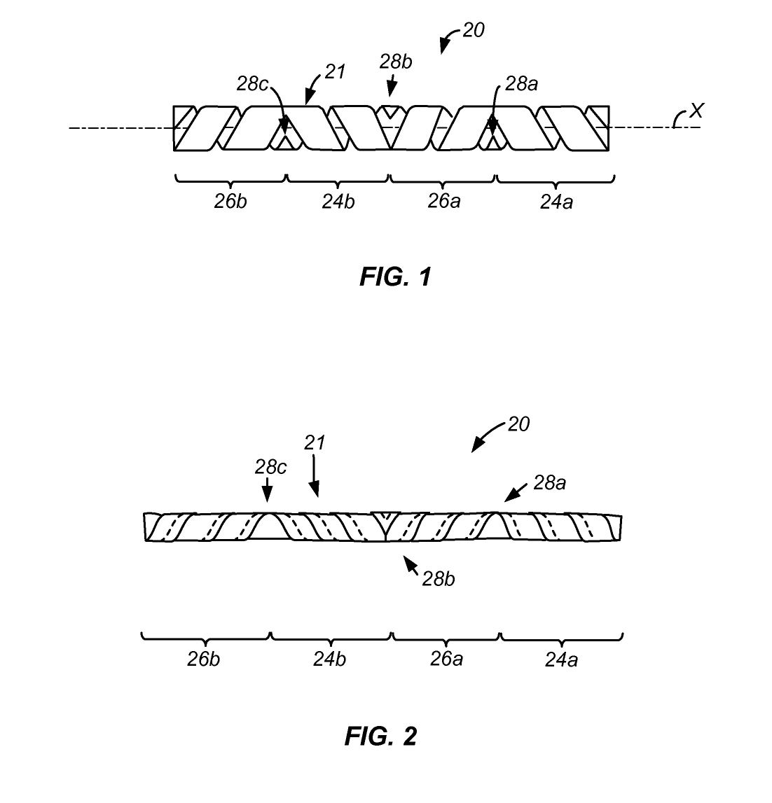

Referring now to FIGS. 1 and 2, a schematic representation of a vascular prosthesis 20 shown in a radially expanded, deployed state and a radially contracted, delivery state, respectively. Vascular prosthesis 20 is constructed from two or more helical portions having at least one change in the direction of rotation of the helices, and being joined at apex portions where the directions of rotation of adjacent helices change. In particular, first (i.e., proximal-most) helical portion 24a has a generally clockwise rotation about longitudinal axis X of prosthesis 20. Helical portion 26a adjoins the distal end of helical portion 24a at apex 28a and has a generally counter-clockwise rotation about longitudinal axis X. Helical portion 24b adjoins the distal end of helical portion 26a at apex 28b, and in turn is coupled to the proximal end of helical portion 26b at apex 28c. As a result of the alternating direction of rotation of the adjoining helical portions 24a, 26a, 24b and 26b of vascular prosthesis 20 includes three apices 28a, 28b and 28c that are oriented such that they point in alternating directions about the circumference of vascular prosthesis 20, generally in planes that are normal to longitudinal axis X of vascular prosthesis 20.

Alternating helical section 21 can be formed from a solid tubular member or sheet comprised of a shape memory material, such as nickel-titanium alloy (commonly known in the art as Nitinol). However, it should be appreciated that alternating helical section 21 may be constructed from any suitable material or processes recognized in the art. The prosthesis may then be laser cut or photoetched, using techniques that are known in the art, to define a specific pattern or geometry in the deployed configuration. Alternating helical section 21 can be cut or etched from the tube or sheet material so that helical portions 24a, 26a, 24b, 26b are integrally formed as a single monolithic body. However, it should be appreciated that separate helical portions may be mechanically coupled, such as by welding, soldering or installing mechanical fasteners to construct alternating helical section 21. An appropriate heat treatment then may be applied to alternating helical section 21 of vascular prosthesis 20 so that the device may be configured to self-deploy from a contracted delivery configuration to the expanded deployed configuration.

Referring now to FIG. 2, the vascular prosthesis 20 is shown in the contracted and partially overlapped, delivery configuration, wherein alternating helical section 21 is in the contracted, reduced diameter state. The vascular prosthesis 20 is placed in the contracted state by winding helical portions 24, 26 about longitudinal axis X. When vascular prosthesis 20 is loaded onto a delivery device, apices 28a and 28c are temporarily retained on an elongate body of a delivery system, and apex 28b and the distal and proximal ends of alternating helical section 21 are rotated relative to the elongate body until vascular prosthesis is in the contracted state as shown. As a result, apices 28a and 28c are wrapped radially inward of the remainder of vascular prosthesis 20 and will be generally referred to herein as “inner apices.” Conversely, apex 28b, which will be generally referred to as an “outer apex,” and the distal and proximal ends of alternating helical section 21 are wrapped radially outward of the remainder of alternating helical section 21.

Consequently, apices 28a and 28c are tightly wound onto the shaft of the delivery catheter and the remainder of each helical portion 24, 26 is wound against the shaft so that each turn of each portion 24, 26 slightly overlaps an adjacent turn. As a result, apex 28b and the distal and proximal ends of alternating helical section 21 are located furthest radially outward on the rolled alternating helical section 21 and are not secured to the delivery device. The overlap of the turns of helical portions 24, 26 is indicated by dashed lines in FIG. 2. The overlapping turns of alternating helical section 21 thus secure apices 28a and 28c when vascular prosthesis 20 is disposed within a delivery system. In addition, the overlapping of turns results in vascular prosthesis 20 having a unique deployment sequence that allows for increased control over its placement. Moreover, the unique configuration of alternating helical section 21 require a delivery system that allows for temporarily retaining the inner apices of alternating helical section 21 at least during loading.

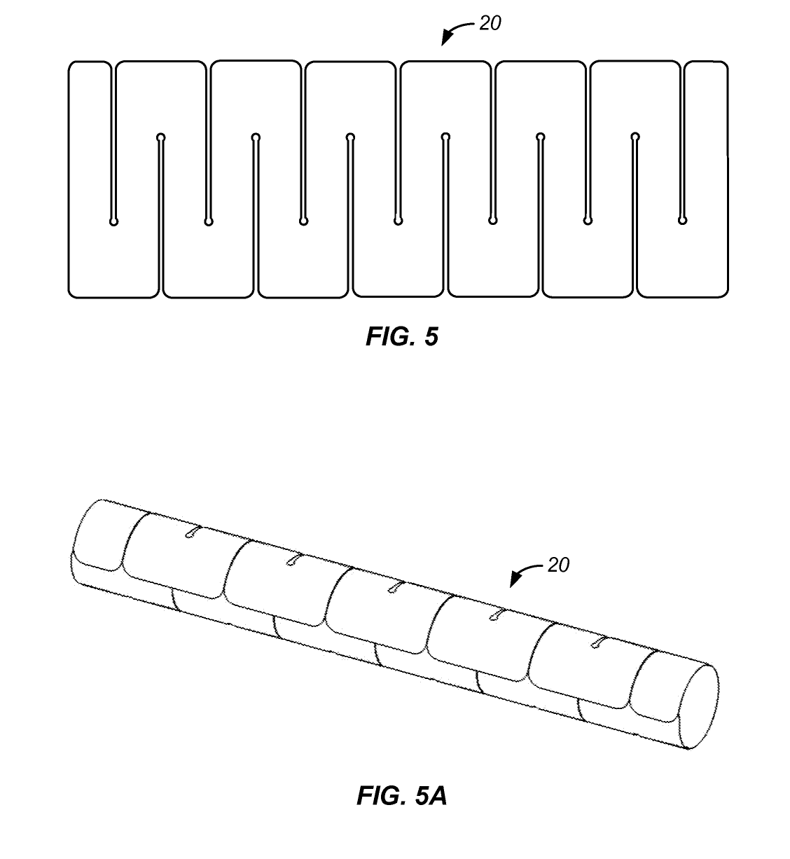

The present invention can be carried out with vascular prosthesis being constructed in a manner other than vascular prosthesis 20. For example, instead of being a ribbon-like material, the vascular prosthesis may be a wire having a round or other cross-sectional shape and may not have overlapping elements. Also, instead of having alternating helical sections, the entire prosthesis may be wound in a single direction. In another example, the vascular prosthesis is not helically wound but may be circumferentially wrapped; see FIGS. 5 and 5A. The prosthesis can alternatively be a radially compressible slotted tube design. In any event, the outer delivery sheath 42 maintains the vascular prosthesis 20 in the radially contracted state.

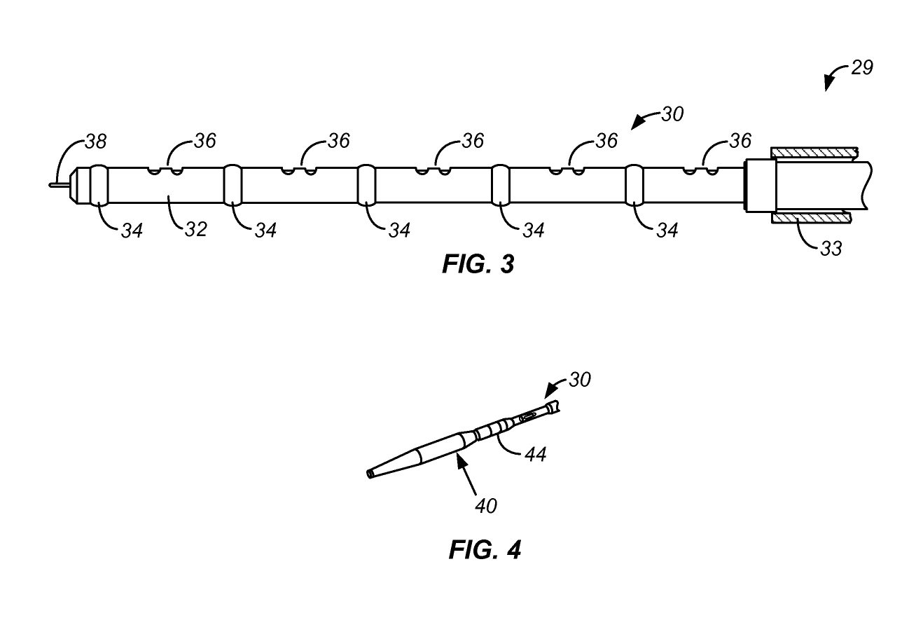

Referring to FIG. 3, one example of a delivery device 29 is shown. Delivery device 29 includes a delivery catheter 30, comprising an inner catheter body 32 and an outer delivery sheath 33 slideably mounted over the inner catheter body. Catheter 30 is the type shown in US patent application publication number US 2008/0021657 A1, the disclosure of which is incorporated by reference. The outer diameter of the inner catheter body 32 may be altered by pads (“bumps”) 34 that extend radially outward from the outer surface of catheter body 32. Pads 34 may be resilient or rigid rings that are coupled to the outer surface of catheter body 32 and spaced from retainers 36. The retainers are designed to hold the vascular prosthesis 20 at apices along one side of the prosthesis (“inner apices”), allowing the prosthesis to be held while the prosthesis is wrapped about the catheter body. The pads 34 may alternatively be designed with a geometry that mates with cavities in the constrained for deployment stent configuration. The catheter body 32 can be constructed from a high-strength resilient material, such as nylon, polyimide or polyetheretherketone (PEEK), so that it is flexible yet durable. The body may further be supported by a metallic matrix such as a braid or coil. Pads 34 may be made from a rigid or resilient material. Alternatively, the pads may be expanded from the inner shaft catheter body material, as one would blow a balloon. Additionally, marker bands to aid in stent position identification may be entrapped during the tip creation by placing them onto the inner shaft catheter prior to the blowing process.

Retainers 36 may be eyelets, notches, or similar structures in catheter body 32. A retaining wire, not shown, may be used to hold the prosthesis 20 to the catheter body 32. The retaining wire may be of a material such as high-strength polymer or Nitinol metallic wire. The retaining wire may run down the primary lumen of the catheter body 32 which may be sized to traverse over a guidewire 38. Alternatively, the retaining wire may be placed in a secondary, small diameter lumen.

Referring to FIG. 4, the inner catheter body 32 preferably includes an atraumatic tip 40 (not shown in FIG. 3), providing a smooth transition to the prosthesis 20. The tip 40 may comprise of a soft, lubricious material including, but not limited to polyether block amide (Pebax), nylon, polytetrafluoroethylene (PTFE), or fluorinated ethylene propylene (FEP). The atraumatic tip 40 may be comprised of a separate component attached to the inner catheter body 32, or the tip may be expanded from the inner catheter body material, as one would blow a balloon. Forming the tip 40 completely from the inner catheter body material reduces or potentially eliminates the concern of a tip breaking off and becoming an embolic risk. Material flow pre or post-blowing may be performed to create the desired tip transition, such as pre-necking the inner shaft material and blowing the entire tip configuration. Additionally, marker bands 44 may be entrapped during the tip creation by placing them onto the inner catheter body prior to the blowing process.

The following deployment mechanisms described apply to any self-expanding prosthesis configuration. The prosthesis may comprise a super-elastic material, such as Nitinol, or any suitable material recognized in the art, including polymers and biodegradable materials. The prosthesis design may consist of an alternating helix pattern as described above, such as a serpentine pattern as depicted in FIG. 5 with circumferential elements connected on alternating ends, or any other self-expanding design. Additionally, these mechanisms may be used with radially self expanding vascular prostheses for which balloon-expansion is used to provide additional deployment force for the vascular prosthesis.

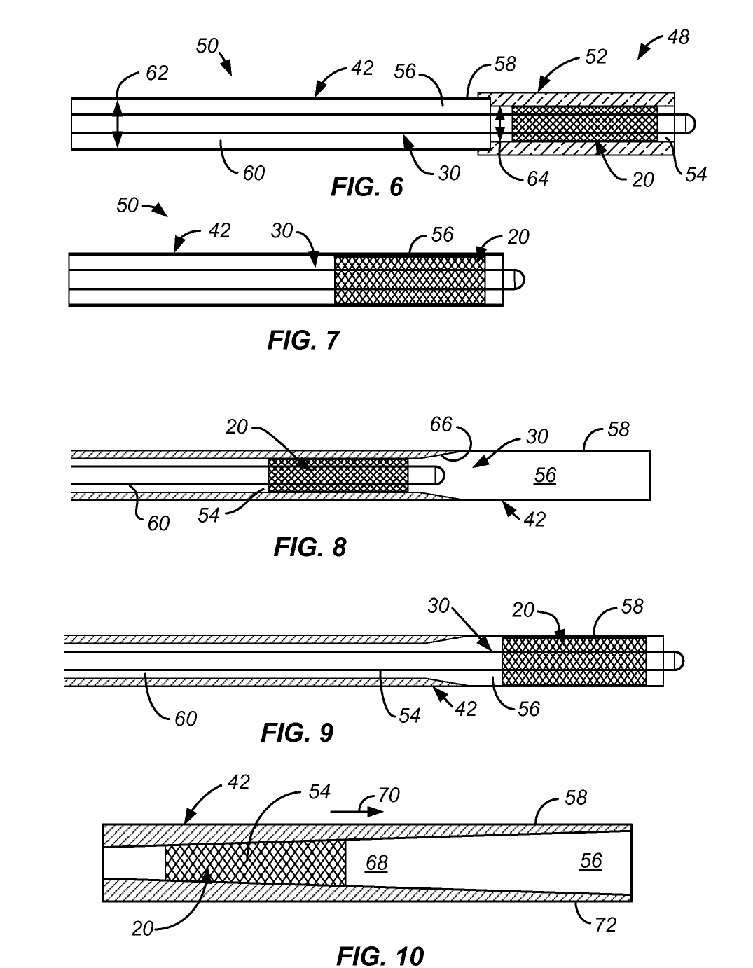

A first example of the invention will be discussed with reference to FIGS. 6 and 7. A delivery system 48 comprises a catheter assembly 50 and a cartridge 52. Catheter assembly 50 comprises an outer delivery sheath 42 and an inner delivery catheter 30. Cartridge 52 acts as an extension of outer delivery sheath 42. A vascular prosthesis 20 is mounted on a distal end of the inner delivery catheter 30. The cartridge 52 provides a temporary vascular prosthesis holding area 54. Vascular prosthesis 20 is typically housed within holding area 54 of cartridge 52 during sterilization and product storage. During clinical use, the prosthesis 20 is transferred from this storage region 54 into the final delivery region 56 in preparation for delivery to the patient site and implantation. This transfer takes place outside of the patient so that the extra force that may be required to transfer vascular prosthesis 20 from cartridge 52 into delivery region 56 of outer delivery sheath 42 is easily managed and does not create a threat of injury to the patient. After placement of vascular prosthesis 20 at delivery region 56, delivery sheath 42 is positioned at the target site within the patient. The short amount of time, typically a matter of minutes, between placement of vascular prosthesis 20 at delivery region 56 and removal of the vascular prosthesis from outer delivery sheath 42, eliminates the additional force that would otherwise be required to deploy the passenger prosthesis for at least two reasons. First, any embedding of the vascular prosthesis caused by plastic creep of the vascular prosthesis pressing against the outer delivery sheath is eliminated. Second, any embedding of the vascular prosthesis into the outer delivery sheath such as can result from sterilization or exposure to other environmental conditions would also be eliminated.

According to this example of this present invention, the vascular prosthesis 20 is captured inside of a constraining apparatus, cartridge 52, which can be separate from the catheter assembly. The vascular prosthesis 20 may be wrapped, then loaded into this temporary cartridge 52 that is sterilized separately from the rest of the device. Before clinical use and deployment, the cartridge 52 with the vascular prosthesis 20 loaded therein, is temporarily attached to the outer delivery sheath 42 and becomes an extension of outer delivery sheath 42. The cartridge 52 may be linked by friction fitting over the outer delivery sheath 42 of the catheter assembly, an o-ring feature, a clamshell design of the cartridge, the use of mating luers, or other appropriate connection mechanism. The cartridge 52 may be made from a lubricious material with sufficient strength to resist the prosthesis 20 from embedding into the inner surface of the cartridge during sterilization. Materials may include PTFE, FEP, polyimide-impregnated PTFE, Delrin®, polyethylene, Nitinol, or a composite such as a PTFE-lined braided tubing. As shown in FIG. 6, the cartridge 52 may be connected to the distal end 58 of the outer delivery sheath 42. Prior to device use, the vascular prosthesis 20 is transferred into a temporary holding area 54 of lumen 60 of outer delivery sheath 42 at the distal end 58 of the sheath to create a loaded catheter assembly 50 as shown in FIG. 7. The lumen 60 preferably has an internal diameter 62 equal to or greater than the cartridge internal diameter 64 of cartridge 52. A change in diameter of just 0.025 mm (0.001″) or 0.05 mm (0.002″) over the stent length is sufficient, but a change 0.076 mm (0.003″) or more is preferable. The cartridge 52 is then removed and the catheter assembly 50 is placed into the vessel. A pusher wire or alternate inner shaft may then be used to transfer the prosthesis 20 from the catheter assembly 50 into the treatment zone.

Alternatively, the cartridge 52 can be attached to the proximal end of the catheter assembly, not shown, and the prosthesis 20 can be transferred distally to its pre-delivery location using a pusher element. In this example, the lumen 60 of outer delivery sheath 42 also preferably has an internal diameter equal to or greater than the cartridge internal diameter. Again, a change in diameter of just 0.025 mm (0.001″) or 0.05 mm (0.002″) over the stent length is sufficient, but a change 0.076 mm (0.003″) or more is preferable. After transfer into the outer delivery sheath 42, the cartridge 52 is removed from the outer delivery sheath 42 and the loaded catheter assembly 50 is placed into the vessel. A pusher wire or alternate inner shaft may then be used to transfer the prosthesis along the catheter assembly into the treatment zone.

Cartridge 52 may be attached to the outer delivery sheath 42 during manufacturing. The cartridge 52 may be linked by a friction-fitting over the outer delivery sheath 42, an o-ring feature, a clamshell design of the cartridge, the use of mating luers, or an alternative mechanism. An inner delivery catheter 30 may be placed through both the outer delivery sheath 42 and the cartridge 52. The vascular prosthesis 20 may be loaded on the inner delivery catheter 30 and transferred into the cartridge 52. The entire system, including the outer delivery sheath 42, inner delivery catheter 30, the vascular prosthesis 20, and the cartridge 52, are then sterilized together or independently. At the clinical site, the vascular prosthesis 20 is transferred into the final sheath location 56 within outer delivery sheath 42 from the cartridge 52. If the cartridge 52 is attached to the proximal end of the outer delivery sheath 42, the vascular prosthesis 20 is pushed into or pulled through the lumen 60 of the outer delivery sheath and the cartridge 52 is removed. If the cartridge 52 is attached to the distal end 58 of the outer delivery sheath 42, the vascular prosthesis 20 may be pulled into the outer delivery sheath 42 from its proximal end using the delivery catheter 30. Alternatively, the vascular prosthesis 20 may be pushed into the outer delivery sheath 42 from the distal end 58 using a tool, such as a pusher wire, to advance the vascular prosthesis 20 through the cartridge 52.

In this example, vascular prosthesis 20 is initially secured to the delivery catheter 30 and can be released from the inner delivery catheter when the vascular prosthesis is outside of the outer delivery sheath 42. However, the invention can also be practiced when the vascular prosthesis 20 is not secured to an inner delivery catheter 30 so that it is pushed out of the distal end 58 of sheath 42 using other mechanisms, such as a pusher wire.

Another example of the invention relates to providing outer delivery sheath 42 with different internal diameters such that, as shown in FIGS. 8 and 9, the temporary vascular prosthesis storage region 54 has a smaller internal diameter than the vascular prosthesis delivery region 56, with regions 54, 56 connected by a tapered transition region 66. Instead of a smoothly tapering transition region 66, the transition region may have a series of smaller internal diameters reaching toward the proximal end. The vascular prosthesis 20 is shown in FIG. 8 constrained in the smaller diameter storage region 54 during sterilization and storage. While the entire vascular prosthesis 20 is shown in FIG. 8 to be located entirely proximal of the delivery region 56, in some examples, only a part of the prosthesis is proximal of delivery region 56. At the clinical site, prior to placement of the catheter assembly into the patient, the vascular prosthesis 20 is advanced into the larger diameter delivery region 56. Advancement of the vascular prosthesis prior to insertion of the delivery system into the patient, allows reduces forces caused by patient anatomical curvatures, accessory device interaction or elevated temperature effects. This allows for a lower deployment force within the vasculature compared to deployment without pre-advancement.

Differences in diameters between the storage region 54 and the delivery region 56 may be as little as 0.025 mm (0.001″) or 0.05 mm (0.002″), but preferably 0.076 mm (0.003″) or greater. The amount of the differences in diameters will depend at least in part upon the materials used, the forces exerted by vascular prosthesis 20 and the subsequent amount of embedding by vascular prosthesis 20 into outer delivery sheath 42. The thickness of stent 20 in the contracted state is preferably greater than the diameter change of the outer delivery sheath 42. This enables a pushing feature on the inner delivery catheter 30 at the proximal end of stent 20 to continuously contact the stent from the cartridge 52 or storage region 54 to the distal end of the delivery region 56. Contracted stent thickness may be achieved through individual wall thickness of stent 20 or the wrapping of stent 20 resulting in multiple layers. Alternatively, the stent 20 may be in intimate contact with the inner delivery catheter 30, e.g. through the use of a retaining wire.

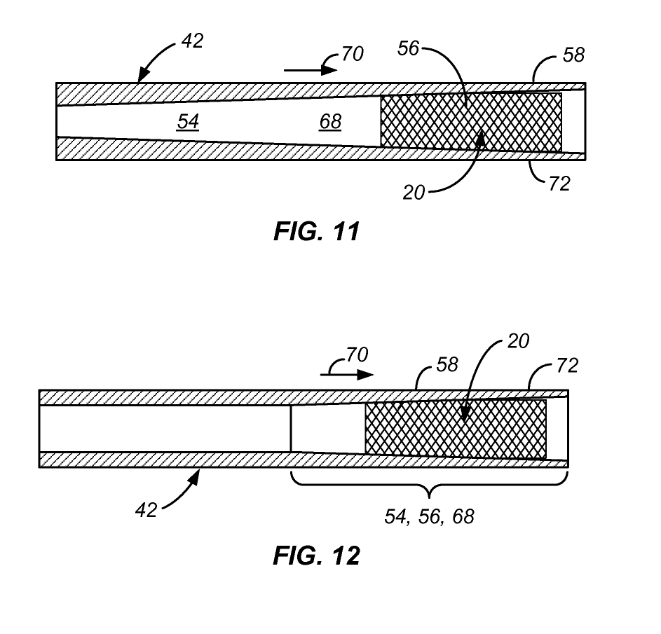

A further example of the invention will be discussed with reference to FIG. 10 and FIG. 11. In this example, the distal end 58 of outer delivery sheath 42 has an outwardly expanding, tapering lumen 68 when considered in a distal direction 70, that is toward the distal tip 72 of outer delivery sheath 42. This section may be a continuous taper, a taper over only a partial length of the stent, or include multiple, stepped diameters. During sterilization and storage, creep of the prosthesis and sheath due to the chronic outward force of the prosthesis, causes discreet lengths of each segment of the prosthesis to grow in diameter. When the prosthesis 20 is pushed through adjacent sections of a straight-profile sheath which have not experienced creep, deployment forces may be very high. Alternately, with the tapered sheath, the prosthesis 20 deploys in the distal direction 70, allowing each segment of the prosthesis to be pushed through a larger opening, thus reducing the forces of deployment. The reduction of deployment force occurs quite quickly after the initial movement of vascular prosthesis 20 in distal direction 70. Vascular prosthesis 20 can be stored and sterilized within proximal region 54 of tapering lumen 68. Vascular prosthesis 20 can then be advanced to the distal region 56 at the clinical site prior to placement of the catheter assembly into the patient. For a tapering lumen 68 having a length of 150 mm, a taper may be as little as 0.025 mm (0.001″) or 0.05 mm (0.002″), but preferably 0.076 mm (0.003″) or greater. This configuration acts to ease the deployment forces for an outer sheath pull-back mechanism. In another example, storage region 54 may be tapered as in FIGS. 10 and 11 but delivery region 56 may have a constant diameter; such constant diameter would typically be equal to or greater than the diameter of storage region 54 at the distal end of the storage region.

In an alternative example, shown in FIG. 12, tapering lumen 68 is relatively short and constitutes both the storage region 54 and the delivery region 56. That is, the storage and delivery regions at least substantially overlap and are therefore generally coextensive. The vascular prosthesis 20 is stored in the vascular prosthesis delivery region 56 through insertion of the delivery system to the patient's target site. Even if a certain amount of embedding had occurred, the taper of delivery region 56 causes the force necessary to push vascular prosthesis 20 out through the distal tip 72 of outer delivery sheath 42 to quickly drop after the initial movement of the vascular prosthesis. That is, after the initial movement of vascular prosthesis 20 distally through the coextensive storage/delivery region 54/56, the average diameter of vascular prosthesis 20 has increased to substantially immediately reduce the ejection force necessary. For a tapering delivery region 56 of this example having a length of slightly longer than the stent length, the overall taper along the length (diameter change) may be as little as 0.025 mm (0.001″) or 0.05 mm (0.002″), but preferably 0.076 mm (0.003″) or greater.

The sheath 42 may include, but is not limited to a metallic matrix of braid or coil, a PTFE liner, and a high-strength laminate layer. There are multiple methods of producing a tapered profile on the inner diameter of the sheath 42. The sheath may be laminated or stretched over a mandrel with the tapered outer diameter profile. The mandrel may be produced via multiple manufacturing methods including, but not limited to centerless grinding or Swiss screw machining. Additionally, stepped internal diameters may be incorporated with the tapered internal diameter. Therefore, tapering region 68 may include a single type of tapered segment or, for example, any combination of straight tapered segments, curved tapered segments and stepped tapered segments. The stepped tapered segments typically include generally axially directed surfaces and generally radially directed surfaces.

To further limit deployment force in the tapering delivery sheath concept exemplified in FIG. 11, the prosthesis 20 may be advanced just prior to device insertion into the patient. This allows the forces associated with prosthesis embedding into the outer shaft to be overcome when outside the patient, while the catheter is straight and at room temperature, when advancement forces will be lowest. As discussed above, such forces may arise as a result of sterilization, shelf life aging, or other changes to temperature and/or humidity. The same procedure may be used with the example of FIG. 12 in which the prosthesis is advanced a short distance through the coextensive storage/delivery region 54/56 just prior to device insertion into the patient in which the prosthesis is advanced a short distance through the coextensive storage/delivery region 54/56 just prior to device insertion into the patient.

The invention has been discussed in terms of smaller diameter storage regions and larger diameter delivery regions. In some examples, such as in FIGS. 9 and 10, the entire storage region will have a smaller diameter than any part of the delivery region. However, in some examples, there may be a portion of the storage region which has a diameter equal to or somewhat greater than a portion of the delivery region; even in such examples the average diameter of the storage region will be smaller than the average diameter of the delivery region so that the diameter of the storage region will be considered smaller than the diameter of the delivery region.

The above descriptions may have used terms such as above, below, top, bottom, over, under, et cetera. These terms may be used in the description and claims to aid understanding of the invention and not used in a limiting sense.

While the present invention is disclosed by reference to the preferred embodiments and examples detailed above, it is to be understood that these examples are intended in an illustrative rather than in a limiting sense. It is contemplated that modifications and combinations will occur to those skilled in the art, which modifications and combinations will be within the spirit of the invention and the scope of the following claims.

Any and all patents, patent applications and printed publications referred to above are incorporated by reference.

What is claimed is:

1. A vascular prosthesis delivery system comprising:
a self expandable vascular prosthesis;
a delivery sheath comprising a main portion defining a lumen, the lumen having a larger diameter delivery region;
the delivery sheath further comprising a removable cartridge portion comprising a lumen, the lumen having a smaller diameter storage region; and
the vascular prosthesis being housed within the storage region and movable into the delivery region for delivery of the vascular prosthesis at a target site within a patient;
whereby the delivery force required to move the vascular prosthesis from the delivery region into the patient can be reduced.

2. The vascular prosthesis according to claim 1, wherein the cartridge portion is removably mounted to a distal end of the main portion.

3. A method for storing a vascular prosthesis and delivering the vascular prosthesis to a target site within a patient comprising:
obtaining a vascular prosthesis comprising a delivery sheath and a vascular prosthesis, delivery sheath comprising a main portion defining a larger diameter delivery region and a removable cartridge portion defining a smaller diameter storage region, the vascular prosthesis stored within the smaller diameter storage region;
moving the vascular prosthesis from the smaller diameter storage region into the larger diameter delivery region in preparation for placing the vascular prosthesis at a target site within a patient; and
moving the vascular prosthesis from the larger diameter delivery region to the target site within the patient.

4. The method according to claim 3, further comprising removing the extension cartridge from a distal end of the main delivery sheath after the first moving step.

5. The method according to claim 3, further comprising removing the extension cartridge from a distal end of the main delivery sheath after the first moving step.

6. A vascular prosthesis delivery system comprising:
a self expandable vascular prosthesis;
a delivery sheath comprising a lumen, the lumen having a smaller diameter storage region and a larger diameter delivery region; and
the vascular prosthesis housed within the storage region and movable into the delivery region for delivery of the vascular prosthesis at a target site within a patient;
whereby the delivery force required to move the vascular prosthesis from the delivery region into the patient can be reduced.

7. The system according to claim 6, wherein the vascular prosthesis comprises at least one of a ribbon-like material comprising at least one circumferential wrap.

8. The system according to claim 6, wherein the delivery sheath comprises a main delivery sheath and an extension cartridge mountable to the main delivery sheath, the extension cartridge comprising the smaller diameter storage region.

9. The system according to claim 8, wherein the extension cartridge is mountable to a distal end of the main delivery sheath.

10. The system according to claim 8, wherein the smaller diameter storage region and the larger diameter delivery region are each generally constant diameter regions.

11. The system according to claim 6, wherein the smaller diameter storage region and the larger diameter delivery region are not each generally constant diameter regions.

12. The system according to claim 6, wherein the storage region defines a tapered lumen, the tapered lumen expanding in diameter in a distal direction.

13. The system according to claim 12, wherein the tapered lumen is a constantly tapered lumen.

14. The system according to claim 12, wherein the tapered lumen is a step tapered lumen with discrete diameters.

15. The system according to claim 6, wherein the storage and delivery regions are generally coextensive and define a tapered lumen expanding in diameter in a distal direction.

16. The system according to claim 6, wherein the delivery region defines a tapered lumen, the tapered lumen expanding in diameter in a distal direction.

17. The system according to claim 6, wherein the entire larger diameter delivery region is distal of the smaller diameter storage region.

18. The system according to claim 6, wherein there is a change in diameter between the storage region and the delivery region of at least 0.076 mm.

19. A vascular prosthesis delivery system comprising:
a self expandable vascular prosthesis;
a delivery sheath comprising a lumen, the lumen having a smaller diameter storage region and a larger diameter delivery region;
the entire larger diameter delivery region being distal of the smaller diameter storage region;
the storage region defining a tapered lumen, the tapered lumen expanding in diameter in a distal direction; and
the vascular prosthesis housed within the storage region and movable into the delivery region for delivery of the vascular prosthesis at a target site within a patient;
whereby the delivery force required to move the vascular prosthesis from the delivery region into the patient can be reduced.

20. The system according to claim 19, wherein the delivery region defines a tapered lumen, the tapered lumen expanding in diameter in a distal direction.

21. A vascular prosthesis delivery system comprising:
a self expandable vascular prosthesis;
a delivery sheath comprising proximal and distal ends and a lumen, the lumen having a smaller diameter storage region and a larger diameter delivery region;
the storage and delivery regions being generally coextensive and defining a tapered lumen extending to the distal end, the tapered lumen expanding in diameter in a distal direction; and
the vascular prosthesis housed within the storage region and movable into the delivery region for delivery of the vascular prosthesis at a target site within a patient;
whereby the delivery force required to move the vascular prosthesis from the delivery region into the patient can be reduced.

22. A method for storing a vascular prosthesis and delivering the vascular prosthesis to a target site within a patient comprising:
obtaining a vascular prosthesis delivery system comprising self expandable vascular prosthesis stored within a smaller diameter storage region of a lumen of a delivery sheath;
moving the vascular prosthesis from the smaller diameter storage region into a larger diameter delivery region of the lumen of the delivery sheath; and
moving the vascular prosthesis from the larger diameter delivery region to a target site within a patient;
whereby the delivery force required to move the vascular prosthesis from the delivery region into the patient can be reduced.

23. The method according to claim 22, further comprising placing the distal end of the delivery sheath at the target site within a patient prior to the vascular prosthesis moving steps.

24. The method according to claim 22, wherein the obtaining step is carried out with the storage region and large diameter delivery region being coextensive.

25. The method according to claim 22, wherein the obtaining step is carried out with the delivery sheath having a constant diameter storage region and a constant diameter delivery region.

26. The method according to claim 22, wherein the obtaining step is carried out with at least the storage region defining a tapered storage region, the tapered storage region expanding in diameter in a distal direction.

27. The method according to claim 22, wherein the obtaining step is carried out with the delivery sheath comprising a main delivery sheath and an extension cartridge mountable to the main delivery sheath, the extension cartridge comprising the smaller diameter storage region.

28. The method according to claim 27, further comprising removing the extension cartridge from the main delivery sheath after the first moving step.

29. The method according to claim 22, wherein the obtaining step is carried out with the smaller diameter storage region defining a tapered smaller diameter storage region, the tapered smaller diameter storage region expanding in diameter in a distal direction, and wherein the first moving step is carried out with the vascular prosthesis being moved from the tapered smaller diameter storage region into the larger diameter storage region.

30. The method according to claim 22, wherein the obtaining step is carried out with the larger diameter delivery region defining a tapered larger diameter delivery region, the tapered larger diameter delivery region expanding in diameter in a distal direction, and wherein the second moving step is carried out with the vascular prosthesis being moved from the tapered larger diameter delivery region to the target site.

31. The method according to claim 22, wherein the obtaining step is carried out so that the storage and delivery regions comprise a generally coextensive storage/delivery region, the storage/delivery region being a tapered storage/delivery region expanding in diameter in a distal direction.
